(19) United States
(12) Patent Application Publication
(10) Pub. No.: US 2023/0257687 A1
(43) Pub. Date: Aug. 17, 2023

(54) NOZZLE FOR FLUID DEPLOYMENT IN BIOREACTORS

(71) Applicant: MERCK PATENT GMBH, Darmstadt (DE)

(72) Inventors: Stefano Berti Perez, West Roxbury, MA (US); Laurent Jourdainne, Stutzheim-Offenhein (FR)

(21) Appl. No.: 18/006,299

(22) PCT Filed: Aug. 4, 2021
(86) PCT No.: PCT/EP2021/071742

§ 371 Date: Jan. 20, 2023

(30) Foreign Application Priority Data
Aug. 7, 2020 (EP) 20305908.4

(51) Int. Cl. C12M 1/00; C12M 1/12; C12M 1/21; C12M 1/36

(52) U.S. Cl. CPC C12M 21/00; C12M 29/06; C12M 25/10; C12M 27/20; C12M 41/02; C12M 41/48

(57) ABSTRACT

A nozzle system for fluid deployment for treating a biological fluid within a bioreactor, having a bioreactor having an internal volume; an adjustable nozzle deployed within the internal volume; a reservoir capable of containing an agent; and a tubing connecting the reservoir and the adjustable nozzle, wherein the adjustable nozzle is capable of being adjusted to distribute a processing agent in a plurality of distribution streams.

### FIELD

Embodiments of the present disclosure relate to bioreactors for processing biological fluids. More particularly, some embodiments disclosed herein include a bioreactor having a nozzle for the delivery of liquids and/or solutions within an inner volume of the bioreactor.

### BACKGROUND

Bioprocessing is the manufacturing of biological fluids, for example, cell culturing, virus and viral vector production, and the like. Bioprocessing is conducted within containers and reactors, which range from 3 liter to 5000 liter reactors. These bioreactors have internal volumes of varying shapes and aspect ratios, which complicates the mixing of components. Many components, e.g., processing agents, may be added during bioprocessing. For example, solutions of adjuvants, cell culture media, pH adjustments, and anti-foam agents may be added during bioprocessing. Typically, these materials are added either via a plurality of ports in the top and bottom of the container/bag, wherein a mixing element distributes them. However, this is an inefficient method for distribution in that the port is typically located along an inner surface of the container and distribution of the materials to where they are needed is often inadequate.

Furthermore, these solutions need to be mixed adequately with the biological fluids within bioreactors. A well-designed mixing system, which include impellers and baffles, provides three basic functions. First, a creation of constant conditions (e.g., nutrients, pH, temperature, etc.) in a homogeneous distribution; second, dispersion of gas, e.g., oxygen, and, third, optimization of heat transfer.

Spargers are used to deliver gases, e.g., oxygen, to a biological process. However, high-shear mixing can create an unfavorable foam on the surface of a biological fluid during bioprocessing. Foam further occurs in bioprocessing due to the introduction of gases into the culture medium and leads to reduced productivity resulting from bursting bubbles that can damage valuable products, a loss of sterility if the foam escapes the bioreactor, or over-pressure if the foam blocks an exit filter. Chemical antifoaming agents, also referred to as “antifoams,” “defoaming agents,” or “defoamers,” are routinely used in bioreactors to reduce the amount of foam that forms on a surface of a biological fluid during bioprocessing. Antifoam agents are known also to negatively affect the biological processes taking place in the bioreactor, i.e., creation and propagation of cells, viruses, viral vectors, etc. For example, as foam is generated, cells rise to the surface and get entrapped in foam, depriving them of nutrients and oxygen, which can lead to cell death. In order to combat foaming, defoaming agents are added, the addition of which causes problems, i.e. quality, time, and expense, in subsequent stages of product development. It has been identified that the distribution of such defoaming agent effects the amount of fluid needed to combat foaming. Defoaming agent can be typically added in two manners. One way is by a drop method from the top of the bioreactor, which includes a peristaltic pump and manual addition when an operator sees foam. The second way is by incorporating a feed tube in the bioreactor, whereby the defoaming agent is also added manually upon identification of foaming.

Present systems and bioreactors do not provide an automatic or semi-automatic in situ solution for achieving efficient foam remediation. Foam level detection and delivery systems are inexpensive but intrusive and may introduce too much defoaming agent. Therefore, a system to non-intrusively detect and remediate a foaming condition by efficiently introducing defoaming agents, as well as other processing agents, in the bioreactor represents an inventive advance in the art.

### BRIEF DESCRIPTIONS OF THE DRAWINGS

FIG. 1 depicts a bioreactor and nozzle system, according to some embodiments described herein;

FIG. 2 depicts a close-up view of the nozzle of FIG. 1, according to some embodiments described herein;

FIG. 3 depicts a close-up view of an alternative nozzle deployment for defoaming agent distribution, according to some embodiments described herein;

FIG. 4 depicts a close-up view of a second alternative nozzle deployment for defoaming agent distribution, according to some embodiments described herein;

FIG. 5 depicts a bioreactor, nozzle system, and monitoring and regulation system according to some embodiments of the disclosure; and

FIG. 6 depicts a window of a bioreactor, and an infrared device for obtaining an image, according to embodiments of the disclosure.

The appended drawings illustrate some embodiments of the disclosure herein and are therefore not to be considered limiting in scope, for the invention may admit to other equally effective embodiments. It is to be understood that elements and features of any embodiment may be found in other embodiments without further recitation and that, where possible, identical reference numerals have been used to indicate comparable elements that are common to the figures.

### SUMMARY OF SOME EMBODIMENTS OF THE DISCLOSURE

The disclosure herein describes some embodiments of systems for liquid level and foam monitoring and regulation in a vessel, such as a biocontainer. For example, a biocontainer with a mixer within upstream bioprocessing applications. In some embodiments, the system comprises a biocontainer, bag, or a bioreactor having a window for monitoring. Non-intrusive instrumentation can then be employed to monitor contents within an inner volume of the biocontainer, bag, or bioreactor. Such non-intrusive systems comprise infrared devices, such as cameras and sensors. Signals and/or images from these devices, which detect a condition (such as a foaming, overpressurized, turbid, low or high temperature, temperature variants in varied regions of an inner volume, e.g., hot and cold spots, leaking, volume level, etc., condition) can be transmitted to a microprocessor and a subsequent feedback signal provided to device for responding to a detected condition. Some embodiments of methods of foam detection disclosed herein comprise infrared (IR) spectroscopy. Infrared radiation is used to excite the molecules of a compound, e.g., a liquid, a foam, etc., which produces a spectrum in the form of colors depending on the temperature of the liquid or foam. The energy absorbed by a molecule as a function of the frequency or wavelength of light determines this spectrum. In the case of foam, because foam absorbs less heat, the energy absorbed by the molecules is much lower, and the spectrum displayed will be in a lower gradient. This spectrum determines how much foam the system has.

Some embodiments of the disclosure comprise a nozzle system for fluid deployment for treating a biological fluid within a bioreactor, the bioreactor having an internal volume; and an adjustable nozzle deployed within the internal volume; a reservoir capable of containing an agent, such as a defoaming agent, a diluted agent or media, cell culture media and other agents used in bioprocessing; and a tubing connecting the reservoir and the adjustable nozzle, wherein the adjustable nozzle is capable of being adjusted to distribute a processing agent in a plurality of distribution streams.

Embodiments of the disclosure also provide nozzle systems and methods for even distribution of defoaming agents to remediate foaming conditions more quickly, and/or less use/no use of defoaming agents, and/or scalability from 3 L to 5000 L bioreactors, and/or low fluid volumes, and/or automated control of defoaming methods.

These and other provisions will become clear from the description, claims, and figures below. Various benefits, aspects, novel and inventive features of the present disclosure, as well as details of exemplary embodiments thereof, will be more fully understood from the following description and drawings. So the manner in which the features disclosed herein can be understood in detail, more particular descriptions of the embodiments of the disclosure, briefly summarized above, may be had by reference to the appended drawings. It is to be noted, however, that the appended drawings illustrate only typical embodiments of this disclosure and are therefore not to be considered limiting of its scope, for the described embodiments may admit to other equally effective bags, bioreactors, films and/or materials. It is also to be understood that elements and features of one embodiment may be found in other embodiments without further recitation and that, where possible, identical reference numerals have been used to indicate comparable elements that are common to the figures.

### DETAILED DESCRIPTION

The disclosure herein describes some embodiments of a system for level monitoring and regulation in a vessel, such as a biocontainer. For example, a biocontainer with a mixer within upstream bioprocessing applications. In some embodiments, the biocontainer is a bag or a bioreactor.

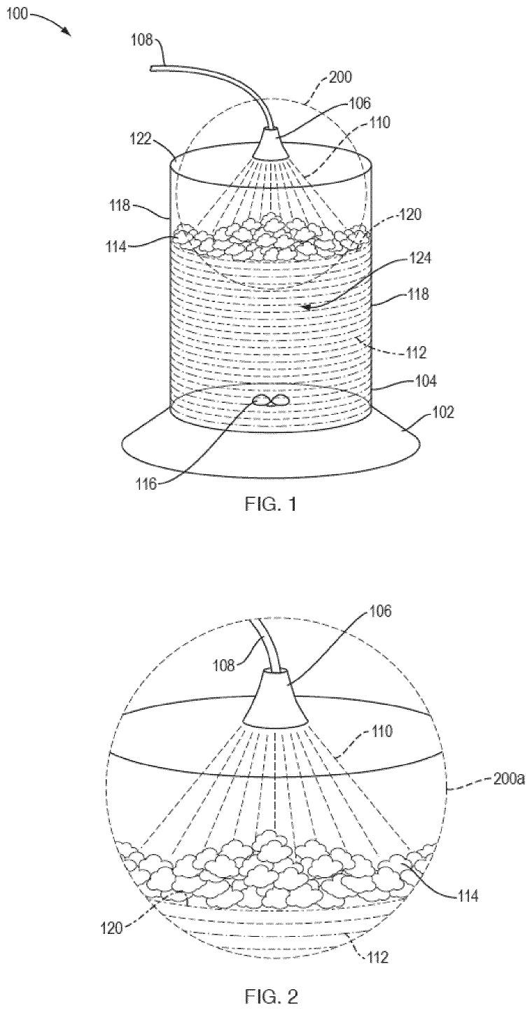

FIG. 1 depicts a bioreactor and nozzle system 100, according to some embodiments described herein. The bioreactor and nozzle system 100 comprises a bioreactor 104, optionally disposed on a base 102. It is to be understood that the bioreactor 104 may also be a single use bioreactor, such as a two- or three-dimensional bag as is known to those in the art. Also, the bioreactor 104 may be a multi-use reactor, for example, a glass, polymeric, or stainless-steel reactor, as is known to those in the art. It is to be further understood that the bioreactor 104 may comprise an inner volume 124 that is, for example, a 3 liter (L) bioreactor or a 3000 L bioreactor, inclusive of all volumes there between. In some embodiments, the inner volume 124 may be larger than 3000 L. A 3 L bioreactor may be made of glass or a transparent plastic. Larger bioreactors may be made of stainless steel and comprise a viewing window. Alternatively, a larger bioreactor may be in the form of a single-use bioreactor comprised of a plastic film.

The bioreactor and nozzle system 100 further comprises a nozzle 106 and distribution tube 108. As shown, the nozzle 106 is disposed on a top surface 122 of the bioreactor 104. However, the nozzle 106 may also be disposed in other regions of the bioreactor 104, such as a sidewall 118, irrespective of whether the bioreactor 104 is a single use or multi-use bioreactor. As shown, the bioreactor 104 contains a biological fluid 112 and foam 114 disposed on a liquid surface 120 of the biological fluid 112. The nozzle 106 is spraying a defoaming agent 110 onto the foam 114. As shown, the nozzle 106 is spraying the foam 114 over a portion of the liquid surface 120. An impeller 116, for mixing the biological fluid 112, is shown disposed on a bottom surface of the bioreactor 104. A gas sparger (not shown) and a baffle (not shown) may also be optionally included within the inner volume 124 of the bioreactor 104, as are known to those in the art.

FIG. 2 depicts a close-up view 200a of the nozzle 106 of FIG. 1, according to some embodiments described herein. As shown, the foam 114 covers the entire liquid surface 120 of the biological fluid 112. The nozzle 106 is spraying a defoaming agent 110 over the entire foam 114.

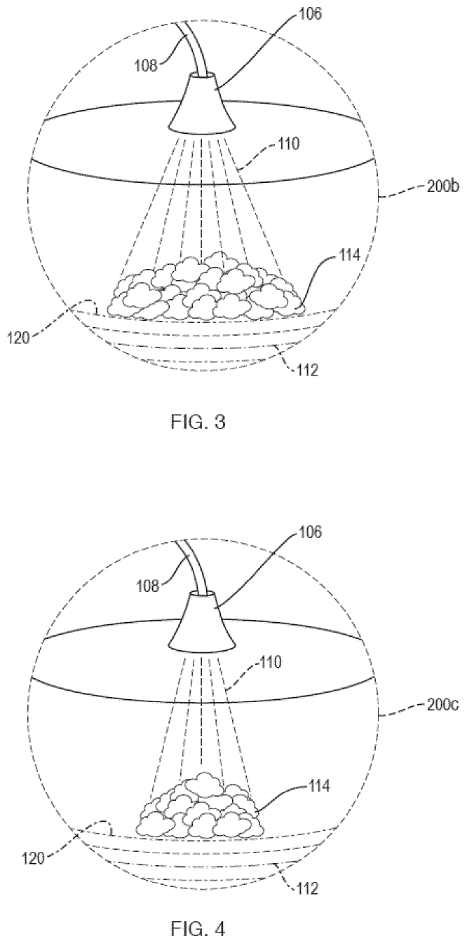

1. FIG. 3 depicts a close-up view 200b of the nozzle 106 of FIG. 1 in an alternative nozzle deployment for defoaming agent 110 distribution, according to some embodiments described herein. As shown, the foam 114 is displaced only on an intermediate area of the liquid surface 120, i.e., the peripheral edges of the liquid surface 120 have little or no foam 114. The nozzle 106 sprays a defoaming agent 110 only approximately on the foam 114 but not on the liquid surface 120. The nozzle 106 may be an adjustable nozzle and/or a nozzle having a less diffuse spraying capacity for optimized spraying. Furthermore, the nozzle 106 may have a manual mechanical adjustment feature or an adjustment feature that is activated by an electric signal. It is to be understood that any adjustable nozzle 106 described herein may be automatically adjusted or manually adjusted.

FIG. 4 depicts a close-up view 200c of the nozzle 106 of FIG. 1 in a second alternative nozzle deployment for defoaming agent 110 distribution, according to some embodiments described herein. As shown, the foam 114 is displaced only on a centralized area of the liquid surface 120, i.e., most of the inner area of the liquid surface 120 and the peripheral edges of the liquid surface 120 have little or no foam 114. The nozzle 106 sprays a focused stream of defoaming agent 110 only on the foam 114 but not on the liquid surface 120. As above, the nozzle 106 may be an adjustable nozzle and/or a nozzle having a less diffuse spraying capacity for optimized spraying. And, as above, the nozzle 106 may have a manual mechanical adjustment feature or an adjustment feature that is activated by an electric signal. With the use of a pump (not shown) in communication with a reservoir (not shown) containing a defoaming agent, the defoaming agent or another liquid solution will travel through tubing 108 to the nozzle 106. Defoaming agent is thereby distributed in an even way where needed within the bioreactor. This can also be achieved using an integrated controlling in the bioreactor with timers/recipes. Solutions of adjuvants, cell culture media, pH adjustments, and other processing components can also be added using a diffuse distribution so mixing is enhanced more quickly. In some embodiments, the nozzle 106 provides distribution having a focused stream, a median stream, and a diffuse stream in a linear or conical angle from 0 to 180 degrees.

A laser-based sensor system may monitor and regulate the level of foam in the bioreactor 104. In some embodiments, the sensor system disclosed is modular, i.e., the sensor system can be successfully implemented with a control device communicating therewith based on a variety of software platforms. In some embodiments, the term modular describes the characteristic of the system described herein as being compatible with different types of bioreactors known in the art. For example, the system may be compatible with multi-use or single-use bioreactors. Alternatively, the system may be compatible with stainless steel bioreactors comprising at least two windows capable of allowing a laser to pass through an inner volume of the bioreactor. Also, in some embodiments, the data processing behind the laser-based sensor system described herein can be simply and fully implementable in presently used software platforms, such as user services platform (USP) software and demand side platform (DSP) software, as is known to those in the art. Some embodiments of the system comprise a non-intrusive sensor, which need not be placed within the inner volume of the bioreactor and does not contact the contents of the biocontainer. The sensor can be in communication with the USP and/or DSP software. For example, the sensor can sense the presence of foam and/or the spread or height of the foam. The sensor USP or DSP can then send a signal to the adjustable nozzle to spray a defoaming agent. The targeted spray or distribution of foam can remediate the foaming condition without spraying an excess amount of defoaming agent. In some embodiments, the system further comprises a collimator. In some embodiments, a photosensor is capable of differentiating the light intensity detected after passing through each of air, foam, and liquid in the vessel. In some embodiments, the photosensor is a photodiode. In some embodiments, the system further comprises a camera capable of capturing live imaging and sensing fluids in the bioreactor. In some embodiments, the system further comprises a collimator and a camera capable of capturing live imaging and sensing fluids in the bioreactor.

In some embodiments, the system is external to the bioreactor 104. In some embodiments, the bioreactor 104 is transparent or translucent, or further comprises at least two windows, wherein a camera or photosensor may be placed to detect fluids within the bioreactor.

Some embodiments described herein provide a method of sensing a level of biological fluid and foam on a surface of the biological fluid within the bioreactor 104 during bioprocessing, the method comprising: splitting a laser light into at least two beams, wherein the at least two beams comprise a first beam and a second beam; directing the first beam through a level of the vessel representing the maximum fill level for contents of the bioreactor 104; wherein the maximum fill level is higher than the level of the contents prior to beginning or continuing bioprocessing; directing the second beam through a level of the bioreactor 104 representing a level for the contents of the bioreactor 104; monitoring the light intensity of the at least two beams by detecting with at least two photosensors, wherein the at least two photosensors comprise a first photosensor and a second photosensor with the first photosensor measuring the light intensity of the first beam and the second photosensor measuring the light intensity of the second beam; activating an alarm when a decrease in light intensity is detected by the first photosensor compared to the light intensity detected by the first photosensor prior to an increase in the level of biological fluid and/or foam in the bioreactor 104; indicating that the level of biological fluid and/or foam in the bioreactor 104 in response to the alarm, and producing a visual or auditory signal and/or sending a signal to a microprocessor to take an action, e.g., activate a pump and/or adjust a nozzle 106 in communication with the pump.

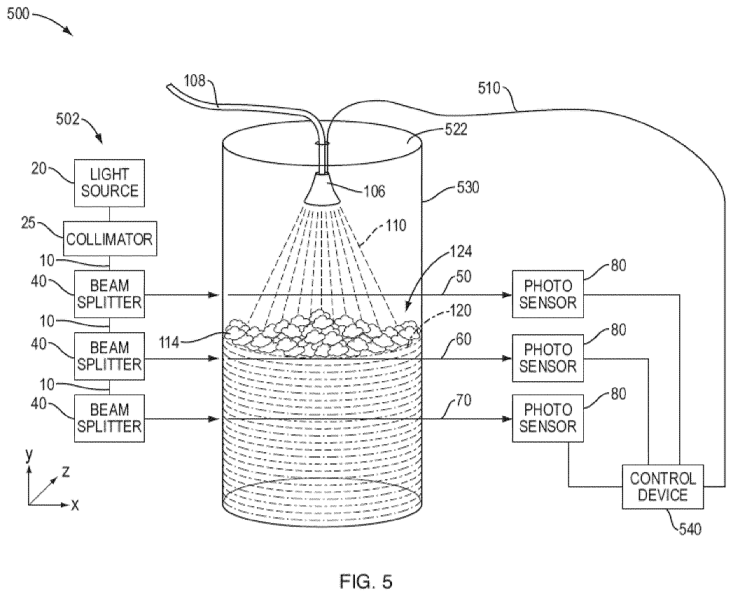

FIG. 5 depicts a bioreactor 530, a nozzle system 106, and a monitoring and regulation system 502, according to embodiments of the disclosure. In some embodiments, a light source 20 emits a laser light 10 with a defined wavelength through the bioreactor 530. In some embodiments, the laser light 10 is split into several beams located at different heights (e.g., the three beams 50, 60, and 70, from highest to lowest liquid height) using a beamsplitter 40 and, optionally, a collimator 25. In some embodiments, a beam 50 is located at a height equivalent to the maximum level for the contents of the bioreactor 530 as appropriate for any given process, as is known to those in the art. As shown, a foam 114 is disposed on top of the liquid level 120. In some embodiments, a beam 60 is located at a height at or near the foam or liquid level 120 of the contents of the bioreactor 530 prior to beginning or continuation of bioprocessing. In some embodiments, a beam 70 is located at a height resulting in the beam 70 travelling through liquid contents of the bioreactor 530.

In some embodiments, the laser light 10 is split into at least two beams. In some embodiments, the laser light 10 is split into at least three beams. For example, the laser light 10 is split into three, four, five, six, seven, eight, or nine beams. In some embodiments, the laser light 10 is split as often as desired. In some embodiments, a plurality of light sources are used to generate the more than one beam (not shown).

In some embodiments, the wavelength of the laser light 10 is within the range of 780 nm to 900 nanometers (nm) and wavelengths therebetween. In some embodiments, the wavelength of the laser light 10 is 780 nm to be close to turbidity standard wavelength (800 nm). In some embodiments, the beam (any of beams 50, 60, and 70) has an elliptical shape section of about 1 mm². In some embodiments, the elliptical shape section of the beam (50, 60, and 70) is less than 1 mm². For example, the elliptical shape section of the beam (50, 60, and 70) is about 0.1, about 0.2, about 0.3, about 0.4, about 0.5, about 0.6, about 0.7, about 0.8, or about 0.9 mm².

In some embodiments, the system comprises more than one beamsplitter 40. For example, the system comprises two, three, four, five, six, or seven beamsplitters 40. In some embodiments, the system comprises three beamsplitters 40. The beamsplitter 40 may comprise a beam diameter within the range of approximately, for e.g., 3 millimeters (mm) to approximately 150 mm. In some embodiments, the beamsplitter 40 has a beam diameter of about 5 mm. For example, the beam diameter is selected from the group consisting of 3 mm, 4 mm, 5 mm, 6 mm, 7 mm, and 8 mm. In some embodiments, the beamsplitter 40 has a reflectance/transmittance (R/T) ratio adjustable between 10/90, 30/70, 50/50, 70/30, and 90/10, and all ranges therebetween.

In some embodiments, each beam is paired with a photosensor 80 to form an optical channel. In some embodiments, two or more optical channels measure continuously and/or simultaneously. In some embodiments, the optical channels are identical to the each other except for the height localization of each. For example, the laser wavelength is the same for each optical channel. In some embodiments, each optical channel includes the same type of photosensor 80. In some embodiments, the height localization of each channel is free before any sensor manufacturing and can be driven by the details of the application of the system.

In some embodiments, an optical channel is formed by one beam (e.g., 50, 60, or 70) and one photosensor 80 located on a same theoretical diameter of the bioreactor 530, resulting in the incident light being fully perpendicular to an axis traversing the circular shape of the bioreactor 530 (normal incidence) to avoid any refraction. Therefore, the transmitted light is measured by a photosensor 80, such as a photodiode. In some embodiments, the photosensor 80 is a silicon-based photodiode or other materials, e.g., germanium, indium gallium arsenide, lead (II) sulfide, and mercury cadmium telluride known to those in the art.

In some embodiments, one mode of operation is integration of a two optical channel laser-based sensor system as described herein into USP equipment. In some embodiments, one optical channel including a beam 60 is located at the liquid surface level (foam channel) 120. In some embodiments, a second optical channel including a beam 50 is located at a reasonable distance from the top of the bag or bioreactor 530 (top channel), i.e., a dual optical channel. In some embodiments, the dual optical channel arrangement of the system functions as a critical level sensor. In some embodiments, an increase in the level of foam 114 in the bioreactor 530 is indicated by a decrease in the light intensity of the beam 50 detected by the photosensor 80. Should the light intensity of the beam 50 decrease to less than a threshold value, the foam 114 has reached a specific height in the bioreactor 530. Then, the level information is fed into a regulation loop 510 for monitoring an operating state of the bioreactor 530.

In some embodiments, the regulation loop 510 is managed by a control device 540, such as a microprocessor or computer, connected to a power supply (not shown), which optionally provides power to the light source(s). In some embodiments, when the control device 540 receives information the level of contents in the bioreactor 530 has reached a critical level, e.g., excessive foam heights, the control device 540 triggers release of a defoaming agent 110 from a conduit 108 in fluid communication with an internal cavity 124 of a single-use or stainless steel biocontainer or bioreactor 530. The bioreactor 530, the nozzle system 106, and the monitoring and regulation system 502 work in conjunction to deliver the defoaming agent 110 to the foam 114 on the liquid surface 120 in a controlled manner via the nozzle 106. In other words, based on the amount of the foam 114 detected by the monitoring and regulation system 502, the nozzle 106 is adjusted commensurately. It is to be understood that any adjustable nozzle 106 described herein may be automatically adjusted or manually adjusted. The nozzle 106 may be adjustable so that the stream of defoaming agent 110 is delivered in a focused stream or in a more diffuse stream, as discussed above. In some embodiments, the nozzle provides distribution having a focused stream, a median stream, and a diffuse stream in a linear or conical angle from 0 to 180 degrees. Furthermore, the amount of the foam 114 may be detected by a staggered array of the light source(s) 20 and photosensor(s) 80. In other words, in some embodiments, the adjustability of the nozzle 106, controlled by a signal sent by the control device 540 to the nozzle 106, depends upon the amount of the foam 114 detected when a plurality of the light source(s) 20 and the photosensor(s) 80 are in differing ‘z’ positions, wherein the beams 50, 60, 70 traverse outer edges of a perimeter of the liquid surface 120 (as opposed to traversing only the center, or near center, of the inner volume 124 of the bioreactor 530).

In some embodiments, four measurements situations occur in a system with at least two optical channels. 1) Light intensities of greater than 0 mA are measured in both optical channels, which means the level of the biological fluid in the bioreactor is low or no foam is detected. 2) A light intensity of 0 mA is measured in the foam channel, and a light intensity of greater than 0 mA is measured in the top channel. These results mean foam or opaque solution is present, but no overflow of the biological fluid has occurred. In some embodiments, anti-foaming agent is added for regulation of the level of the biological fluids in the bioreactor. 3) A light intensity of greater than 0 mA is measured in the foam channel, and a light intensity of 0 mA is measured in the top channel. For a transparent solution, the foam level is too high, and an alarm may be activated or a signal sent. 4) When 0 mA light intensity is measure in both optical channels, the foam or liquid level is too high in the bioreactor. In some embodiments, an alarm is activated. In some embodiments, a defoaming agent is added to the inner volume of the bioreactor. In some embodiments, the camera is a forward-looking infrared (FLIR) camera. The FLIR camera can be a handheld camera or may be mounted to a bioreactor, bag, or biocontainer. In some embodiments, the FLIR camera may be electrically and/or electronically paired with an adaptor for sending a wireless signal to a microprocessor, an iPhone®, an Android® phone, a computer, etc., to send a feedback signal to a device for introducing or delivering an agent, such as an antifoam agent, into a biocontainer, bag, or bioreactor. Any camera, laser, photodiode or sensor described herein can be employed inline for constant and continuous monitoring. Alternatively, the camera, laser, photodiode or sensor described herein can monitor a process on an intermittent basis.

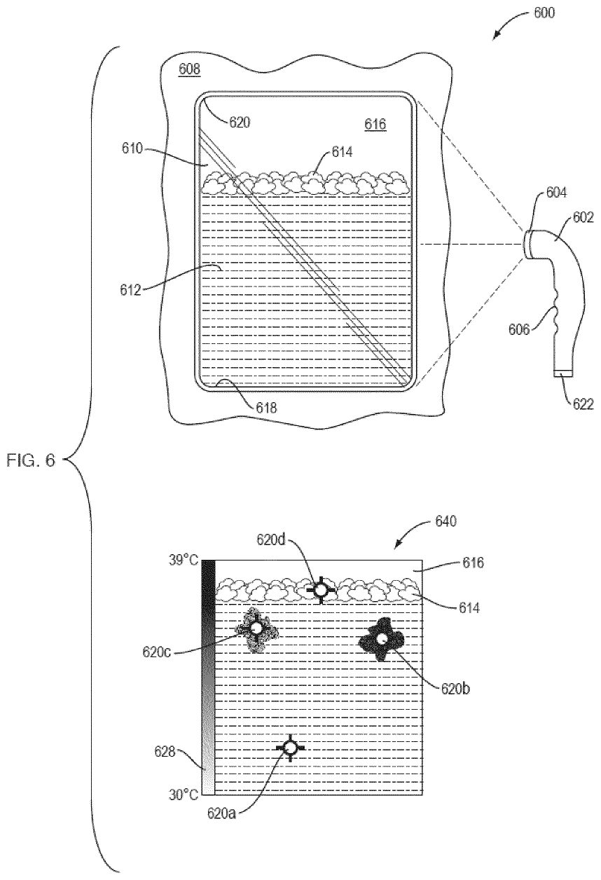

FIG. 6 depicts a system 600, comprising a window 610 of a bioreactor 608, and an infrared device 602 for obtaining an image 640, according to embodiments of the disclosure. The system 600 comprises a bioreactor 608 (shown in a cutaway view) having a window 610. It is to be understood that the bioreactor 608 may be a stainless steel bioreactor having a window 610 or a single use bioreactor made of a polymeric film or composite, wherein a window 610 is placed therein. Alternatively, the bioreactor 608 may be a multi-use bioreactor made of a transparent plastic or glass. As shown, the window 610 has a top region 620 and a lower region 618. In general, the window 610 is placed in a top half of the bioreactor 608. The bioreactor 608 is shown having a fluid 612, such as a biological liquid, therein. On top of the fluid 612 is a foam 614. A space 616, which is a gas or mixtures of gases, such as air, is shown above the foam 614. A thermographic camera 602, such as an infrared camera, such as a forward looking infrared camera, is shown taking an image through a lens 604. The camera 602 may be a handheld camera or it can be mounted on a bioreactor or equipment (not shown). In some embodiments, the camera 602 is a handheld camera, optionally having a pistol grip 606. Also, the camera 602 may have a device 622 for transmitting a signal to a microprocessor. For example, the device 622 may be a wireless signal transmitter. In some embodiments, the device 622 is a USB Wi-fi adaptor for wireless connection between the thermographic camera 602 and a microprocessor and/or a nozzle, as described above.

The image 640 taken by the thermographic camera 602 is shown having a thermographic scale 628 from 30° C. to 39° C. The thermographic scale is meant to show a lighter gray at the lower temperature of the 30-39° C. spectrum and a darker black at higher temperatures. The thermographic camera 602 can take images to monitor biological processes. In biological processes, there are often relative hot and spots within a bioreactor. For example, spot 620a can be viewed as a cold spot, at approximately 31° C. Spot 620c might be a moderate temperature, for e.g., 35° C. Spot 620b might be a hotter spot, for e.g., 38° C. Note that spots 620a, 620b, and 620c are all within liquid contents. Such disparities in temperature can exists because of inadequate mixing. If the thermographic camera 602 detects such a condition, a signal from the camera 602 can be sent to a microprocessor (not shown) for increasing a mixing action, such as by an impeller. It is useful to keep mixing to a minimum because the shear effects of mixing can damage the products, e.g., viral vectors, cells, mAbs, and the like, being processed in a biological process. Also, the camera 602 can detect a temperature variance due to a foam 614. A foam 614 will not have the same temperature as a liquid 612 it is disposed upon. Typically, the foam 614 will be cooler. As shown, the foam is approximately 32° C. Foaming conditions rob cells of oxygen and nutrients, leading to cell death. Accordingly, it is best to avoid foam. When the camera 602 detects foam because of the temperature difference, a signal can be sent from the camera 602 to a microprocessor or device to deliver an antifoaming agent to the bioreactor. In some embodiments, that antifoaming agent is delivered through a nozzle, as described above. Furthermore, the camera 602, if taking an image, detects no temperature differences, i.e., this could indicate that the bioreactor is leaking, and so, seeing only a temperature associated with air because no liquid or foam is present. In such instances, a signal could be sent, triggering an audio or visual alarm, so that appropriate action could be taken.

The FLIR system may further comprise a nozzle or an adjustable nozzle in communication with the bioreactor. A method for detecting a temperature of a fluid, a foam, or a gas using the FLIR system incudes generating a signal, transmitting the signal to a microprocessor, and sending a signal for a device in communication with the bioreactor.

Unless defined otherwise, all technical and scientific terms used herein have the same meaning as commonly understood by one of ordinary skill in the art to which this invention pertains.

The term bioreactor, as used herein, refers to any manufactured or engineered device or system that supports a biologically active environment. In some instances, a bioreactor is a vessel in which a cell culture process is carried out which involves organisms or biochemically active substances derived from such organisms. Commonly used bioreactors are typically cylindrical and are made of stainless-steel or are a flexible bag comprising polymeric films, wherein the films are translucent or transparent.

The term bioprocessing, as used herein, refers to any application of the biological systems of living cells or their components, such as bacteria, enzymes, or chloroplasts, viruses, and cells to obtain a target product. Bioprocessing may encompass upstream and downstream bioprocessing. Upstream bioprocessing includes cell culturing methods and products.

The terms, laser or light source, as used herein, refers to a device capable of producing a coherent beam of light.

The term, photosensor, as used herein, refers to a device capable of detecting light and measuring the light intensity of a beam. The term photosensor includes, e.g., an electronic component that detects the presence of visible light, infrared transmission (IR), and/or ultraviolet (UV) energy.

### EXAMPLES

### Example 1. Differentiating Materials

Transmitted light intensities were measured by a photodiode for the conditions described in Table 1.

TABLE 1
Light Intensities Measured under Various Conditions

| | Intensity |
|---|---|
| Condition | [mA] |
| Through air (baseline) | 302 mA |
| Through the bioreactor + air (above the solution) | 273 mA |
| Through bioreactor + solution (biological fluid) | 184 mA |
| Through bioreactor + thick foam | 0 mA |
| Through bioreactor + light foam | 29 mA |

The results in Table 1 provide show the photodiode was slightly sensitive to the amount of foam, and the photodiode was sensitive enough to the optical index of the medium to differentiate among the different conditions. Therefore, the system was observed to discriminate among air, foam, and solution in the bioreactor.

### EQUIVALENTS

All ranges for formulations recited herein include ranges therebetween and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4, or 3.1 or more.

Reference throughout this specification to “one embodiment,” “certain embodiments,” “one or more embodiments,” “some embodiments,” or “an embodiment” indicates that a feature, structure, material, or characteristic described is included some embodiments of the disclosure. Therefore, the appearances of the phrases such as “in one or more embodiments,” “in certain embodiments,” “in one embodiment,” “some embodiments,” or “in an embodiment” throughout this specification are not necessarily referring to the same embodiment. As used herein, the singular forms of “a”, “an,” and “the” include plural forms unless the context dictates otherwise.

Publications of patent applications and patents and other non-patent references, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

1. A nozzle system for fluid deployment for treating a biological fluid within a bioreactor, comprising:
a bioreactor having an internal volume;
an adjustable nozzle deployed within the internal volume;
a reservoir capable of containing an agent, a diluted agent or media; and
a tubing connecting the reservoir and the adjustable nozzle, wherein the adjustable nozzle is capable of being adjusted to distribute a processing agent in a plurality of distribution streams.

2. The nozzle system of claim 1, wherein the adjustable nozzle is capable of distribution streams that provide a focused stream, a median stream, and a diffuse stream in a linear or conical angle from 0 to 180 degrees.

3. The nozzle system of claim 1, wherein the adjustable nozzle is automatically adjusted or manually adjusted.

4. The nozzle system of claim 1, further comprising a sparger.

5. The nozzle system of claim 1, further comprising an impeller.

6. The nozzle system of claim 1, further comprising a baffle.

7. The nozzle system of claim 1, wherein the processing agent is a defoaming agent.

8. The nozzle system of claim 1, further comprising a monitoring and regulation system for the bioreactor, the monitoring and regulation system comprising:
a light source capable of emitting laser light through the bioreactor;
a beamsplitter capable of splitting the laser light into more than one beam, wherein each of the more than one beam is at a different height of the bioreactor; and
a plurality of photosensors capable of measuring a light intensity of each beam, wherein each more than one photosensor corresponds to one laser beam to form an optical channel or a camera capable of capturing live imaging and sensing fluids in the bioreactor.

9. The nozzle system of claim 8, wherein the bioreactor is a multi-use bioreactor having a window or a single use bioreactor comprising a polymeric film.

10. The nozzle system of claim 9, the bioreactor further comprising a mixing system.

11. The nozzle system of claim 10, wherein the mixing system is capable of being used in at least one of upstream bioprocessing applications and downstream bioprocessing applications.

12. The nozzle system of claim 8, wherein the monitoring and regulation system is capable of measuring a level of contents within the bioreactor.

13. The nozzle system of claim 12, further comprising an alarm capable of being activated by the contents within the internal volume reaching a critical level within the bioreactor.

14. The nozzle system of claim 12, wherein the contents comprise a liquid that includes a solution, a biological fluid, or foam on a liquid surface.

15. (canceled)

16. (canceled)

17. (canceled)

18. The nozzle system of claim 8, wherein each photosensor of the plurality of photosensors is capable of differentiating the light intensity detected after passing through each of air, foam, and liquid in the bioreactor.

19. (canceled)

20. (canceled)

21. A method for treating a biological fluid, comprising:
providing a nozzle system for fluid deployment for treating a biological fluid within a bioreactor, further comprising:
a bioreactor having an internal volume;
an adjustable nozzle deployed within the internal volume;
a reservoir capable of containing an agent; and
a tubing connecting the reservoir and the adjustable nozzle, wherein the adjustable nozzle is capable of being adjusted to distribute a processing agent in at least one of a plurality of distribution streams.

22. The method of claim 21, wherein a defoaming agent is added to contents of the bioreactor focused to only an area covered by a foam.

23. The method of claim 21, further comprising a monitoring and regulation system for the bioreactor, the monitoring and regulation system comprising:
a light source capable of emitting laser light through the bioreactor,
a beamsplitter capable of splitting the laser light into more than one beam, wherein each of the more than one beam is at a different height of the bioreactor, and
a plurality of photosensors capable of measuring a light intensity of each beam, wherein each more than one photosensor corresponds to one laser beam to form an optical channel or a camera capable of capturing live imaging and sensing fluids in the bioreactor.

24. The method of claim 23, wherein the monitoring and regulation system detects a location of a foam on a surface of the biological fluid contained within the internal volume.

25. The method of claim 24, wherein a microprocessor sends a signal to the adjustable nozzle to spray a stream of the agent on the foam.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)
